(19) United States
(12) Patent Application Publication
(10) Pub. No.: US 2022/0031389 A1
(43) Pub. Date: Feb. 3, 2022

(54) CATHETER FOR PERI-VASCULAR FLUID INJECTION

(71) Applicant: Ablative Solutions, Inc., San Jose, CA (US)

(72) Inventors: David R. Fischell, Fair Haven, NJ (US); Darrin James Kent, Murrieta, CA (US)

(21) Appl. No.: 16/945,077

(22) Filed: Jul. 31, 2020

(51) Int. Cl. A61B 18/14

(52) U.S. Cl. CPC A61B 18/1492; A61B 2018/00577; A61B 18/1477

(57) ABSTRACT

An intravascular catheter for peri-vascular and/or peri-urethral tissue ablation includes multiple needles advanced through supported guide tubes which expand around a central axis to engage the interior surface of the wall of the renal artery or other vessel of a human body allowing the injection an ablative fluid for ablating tissue, and/or nerve fibers in the outer layer or deep to the outer layer of the vessel, or in prostatic tissue. Applications include renal denervation for the treatment of hypertension, atrial fibrillation, congestive heart failure, tissue ablation for COPD, BPH and prostate cancer and prevention of restenosis after balloon angioplasty or stent implantation and other disorders.

### FIELD

Some aspects of the disclosure are applicable to the field of devices to advance a needle like structure for injection a fluid into a volume tissue outside of the inside wall of a target vessel of a human body. Applications include renal denervation for the treatment of hypertension, atrial fibrillation, congestive heart failure, tissue ablation for COPD, BPH and prostate cancer and prevention of restenosis after balloon angioplasty or stent implantation and other disorders.

### BACKGROUND

Since the 1930s it has been known that injury or ablation of the sympathetic nerves in or near the outer layers of the renal arteries (Renal Denervation) can dramatically reduce high blood pressure. As far back as 1952, alcohol has been used for tissue ablation based renal denervation in animal experiments. Specifically Robert M. Berne in “Hemodynamics and Sodium Excretion of Denervated Kidney in Anesthetized and Unanesthetized Dog” Am J Physiol, October 1952 171:(1) 148-158, describes painting alcohol on the outside of a dog's renal artery to produce denervation.

Because of the similarities of anatomy, for the purposes of this disclosure, the term target vessel will refer here to the renal artery, for hypertension or congestive heart failure (CHF) applications and the urethra for BPH and prostate applications.

Recent technology for renal denervation include energy delivery devices using radiofrequency or ultrasound energy, such as Simplicity® RF ablation catheter from Medtronic, the ultrasound ablation based system from Recor and the Peregrine® chemical denervation catheter from Ablative Solutions.

There are a number of limitations of the Simplicity® system for RF energy delivery as is does not allow for efficient circumferential ablation of the renal sympathetic nerve fibers. If circumferential RF energy were applied in a ring segment from within the renal artery (energy applied at intimal surface to kill nerves in the outer adventitial layer) this could lead to even higher risks of renal artery stenosis from the circumferential and transmural thermal injury to the intima, media and adventitia. Finally, the “burning” of the interior wall of the renal artery using RF ablation can be extremely painful. The long duration of the RF ablation renal denervation procedure requires sedation and, at times, extremely high doses of morphine or other opiates, and anesthesia close to general anesthesia, to control the severe pain associated with repeated burning of the vessel wall. Thus, there are numerous and substantial limitations of the current approach using RF-based renal sympathetic denervation. Similar limitations apply to ultrasound or other energy delivery techniques.

The Bullfrog® micro infusion catheter described by Seward et al in U.S. Pat. Nos. 6,547,803 and 7,666,163, which uses an inflatable elastic balloon to expand a single needle against the wall of a blood vessel, could be used for the injection of a chemical ablative solution such as alcohol but it would require multiple applications as those patents do not describe or anticipate the circumferential delivery of an ablative substance around the entire circumference of the vessel. The greatest number of needles shown by Seward is two and the two needle version of the Bullfrog® would be hard to miniaturize to fit through a small guiding catheter to be used in a renal artery. If only one needle is used, controlled and accurate rotation of any device at the end of a catheter is difficult at best and could be risky if the subsequent injections are not evenly spaced. This device also does not allow for a precise, controlled and adjustable depth of delivery of a neuroablative agent. This device also may have physical constraints regarding the length of the needle that can be used, thus limiting the ability to inject agents to an adequate depth, particularly in diseased renal arteries with thickened intima. Another limitation of the Bullfrog® is that inflation of a balloon within the renal artery can induce possible late vessel stenosis due to balloon injury of the intima and media of the artery, as well as causing endothelial cell denudation.

Jacobson and Davis in U.S. Pat. No. 6,302,870 describe a catheter for medication injection into the interior wall of a blood vessel. While Jacobson includes the concept of multiple needles expanding outward, each with a hilt to limit penetration of the needle into the wall of the vessel, his design depends on rotation of the tube having the needle at its distal end to allow it to get into an outward curving shape. The hilt design shown of a small disk attached a short distance proximal to the needle distal end has a fixed diameter which will increase the total diameter of the device by at least twice the diameter of the hilt so that if the hilt is large enough in diameter to stop penetration of the needle, it will significantly add to the diameter of the device. Using a hilt that has a greater diameter than the tube, increases the device profile, and also prevents the needle from being completely retracted back inside the tubular shaft from which it emerges, keeping the needles exposed and potentially allowing accidental needlestick injuries to occur. For either the renal denervation or atrial fibrillation application, the length of the needed catheter would make control of such rotation difficult. In addition, the hilts, which limit penetration, are a fixed distance from the distal end of the needles. There is no built in adjustment on penetration depth which may be important if one wishes to selectively target a specific layer in a vessel or if one needs to penetrate all the way through to the volume past the adventitia in vessels with different wall thicknesses. Jacobson also does not envision use of the injection catheter for denervation. Finally, FIG. 3 of the Jacobson patent shows a sheath over expandable needles without a guide wire and the sheath has an open distal end which makes advancement through the vascular system more difficult. Also, because of the hilts, if the needles were withdrawn completely inside of the sheath they could get stuck inside the sheath and be difficult to push out.

As early as 1980, alcohol has been shown to be effective in providing renal denervation in animal models as published by Kline et al in “Functional re-interiorvation and development of supersensitivity to NE after renal denervation in rats”, American Physiological Society 1980:0363-6110/80/0000-0000801.25, pp. R353-R358. Kline states that “95% alcohol was applied to the vessels to destroy any remaining nerve fibers. Using this technique for renal denervation, we have found renal norepinephrine concentration to be over 50% depleted (i.e. <10 mg/g tissue) two weeks after the operation.” Again in 1983 in the article “Effect of renal denervation on arterial pressure in rats with aortic nerve transaction” Hypertension, 1983, 5:468-475, Kline again publishes that a 95% alcohol solution applied during surgery is effective in ablating the nerves surrounding the renal artery in rats. Drug delivery catheters such as that by described by Jacobson which are designed to inject fluids at multiple points into the wall of an artery have existed since the 1990s.

McGuckin in U.S. Pat. No. 7,087,040 describes a tumor tissue ablation catheter having three expandable tines for injection of fluid that exit a single needle. The tines expand outward to penetrate the tissue. The McGuckin device has an open distal end that does not provide protection from inadvertent needle sticks from the sharpened tines. In addition, the McGuckin device depends on the shaped tines to be of sufficient strength so that they can expand outward and penetrate the tissue. To achieve such strength, the tines would have to be so large in diameter that severe extravascular bleeding would often occur when the tines would be retracted back following fluid injection for a renal denervation application. There also is no workable penetration limiting mechanism that will reliably set the depth of penetration of the distal opening from the tines with respect to the interior wall of the vessel, nor is there a preset adjustment for such depth. For the application of treating liver tumors, the continually adjustable depth of tine penetration may make sense since multiple injections at several depths might be needed. However, for renal denervation, the ability to accurately adjust the depth or have choice of penetration depth when choosing the device to be used is important so as to not infuse the ablative fluid too shallow and injure the media of the renal artery or too deep and thus miss the nerves that are in the adventitial and peri-adventitial layers of the renal artery.

Although alcohol has historically been shown to be effective as a therapeutic agent for renal denervation and is indicated by the FDA for use in the ablation of nerves, there is need for an intravascular injection system specifically designed for the peri-vascular circumferential ablation of sympathetic nerve fibers in the outer layers around the renal arteries with sufficient penetration depth to accommodate variability in vessel wall thicknesses and to account for the fact that many renal artery nerves are situated at some distance outside of the artery's adventitia.

In U.S. Pat. No. 9,056,185, issued Jun. 16, 2015, U.S. Pat. No. 9,179,962, issued Nov. 10, 2015, U.S. Pat. No. 9,254,360, issued Feb. 9, 2016, U.S. Pat. No. 9,301,795, issued Apr. 5, 2016, U.S. Pat. No. 9,320,850, issued Apr. 26, 2016, U.S. Pat. No. 9,526,827, issued Dec. 27, 2016, U.S. Pat. No. 9,539,047, issued Jan. 10, 2017, U.S. Pat. No. 9,554,849, issued Jun. 3, 2014, U.S. Pat. No. 9,795,441, issued Oct. 24, 2017, U.S. Pat. No. 10,118,004, issued Nov. 6, 2018, U.S. Pat. No. 10,226,278, issued Mar. 12, 2019, U.S. Pat. No. 10,350,392, issued Jul. 16, 2019, U.S. Pat. No. 10,405,912, issued Sep. 10, 2019, U.S. Pat. No. 10,485,951, issued Nov. 26, 2019, and U.S. Pat. No. 10,576,246, issued Mar. 3, 2020, which are hereby incorporated by reference in their entirety, Fischell et al. show multiple embodiments of a fluid delivery catheter for injection of a fluid into the peri-vascular space of a vessel of a human body. The fluid delivery catheter can include guide tubes and injector tubes with distal needles. Mechanisms shown by Fischell et al. in U.S. Pat. No. 9,931,046, issued Apr. 3, 2018, U.S. Pat. No. 9,949,652, issued Apr. 24, 2018, U.S. Pat. No. 10,022,059, issued Jul. 17, 2018, U.S. Pat. No. 10,420,481, issued Sep. 24, 2019, and U.S. Pat. No. 10,517,666, issued Dec. 31, 2011, which are hereby incorporated by reference in their entirety, can be used to advance electrodes with or without fluid injection capability into and beyond the inside wall of a target vessel for nerve sensing, electrical stimulation and energy based tissue ablation.

Together, these two groups of patents form the “Fischell Patents” for reference throughout this specification and are hereby incorporated by reference in their entirety. In some embodiments described therein, the Fischell Patents can use needle guiding elements in the form of guide tubes to support the advancement and penetration through the inside wall of a target vessel of needles/wires with sharpened distal ends. Such a structure can be important to allow use of small diameter needles/wires that will not cause blood loss when retracted for use in a blood vessel.

Certain catheter embodiments also do not discuss changes to catheter design or other means to best allow access of the catheter from radial artery access which is known to reduce bleeding complications.

The use of 3 hypotubes in certain catheter designs can make the majority of the length of the catheter too stiff to be placed in anything but a very long package that may be less desirable than a catheter that can be wound into a spiral package.

Some designs describe how to reduce the dead space in the fluid injection lumens by inserting a wire into the injector tubes but do not mention methods to reduce the luminal volume in the primary fluid injection lumen that runs the majority of the length of the catheter.

Certain catheters describe catheters that can measure nerve activity but lack the capability to take other important physiological measurements that could be accomplished and would be of value once the catheter is placed into the target vessel.

Prior art methods also describe the use of the prior art catheters with a first step of priming the injection lumen with saline. While this priming is important to remove air from the injection lumen, priming with saline will dilute the ablative fluid that follows in its injection into peri-vascular tissue for the renal denervation application.

Fischell et al. in U.S. Pat. No. 10,485,951, issued Nov. 26, 2019, and incorporated by reference herein, describe techniques for packaging a fluid injection catheter with needles and guide tubes/needle guiding elements. Some embodiments as described herein kits are provided that include items important for use in a renal denervation procedure. Also it is highly desirable in certain cases for there to be additional “spare part” mini-kits of components that can assist in the treatment of multiple vessels or more complex situations.

Throughout this specification any of the terms ablative fluid, ablative solution and/or ablative substance will be used interchangeably to include a liquid or a gaseous substance delivered into a volume of tissue in a human body with the intention of damaging, killing or ablating nerves or tissue within that volume of tissue.

Also throughout this specification, the term inside wall or interior surface applied to a blood vessel, vessel wall, artery or arterial wall mean the same thing which is the inside surface of the vessel wall inside of which is the vessel lumen. Also the term injection egress is defined as the distal opening in a needle from which a fluid being injected will emerge. With respect to the injection needle, either injection egress or distal opening may be used here interchangeably.

The terminology “deep to” a structure is defined as beyond or outside of the structure so that “deep to the adventitia” refers to a volume of tissue outside of the adventitia of an artery.

The term peri-vascular refers to the volume of tissue outside of the inside wall of a target vessel. For an artery this includes the media, external elastic lamina, adventitia and peri-advential tissue.

In some embodiments, a catheter for fluid delivery to a volume of tissue in outside of the inside wall of a target vessel in a human body is provided. The catheter can include a catheter body comprising a catheter fluid injection lumen. The catheter can include a central axis extending in a longitudinal direction. The catheter can include a distal portion comprising at least one guide tube comprising a distal end. In some embodiments, the at least one guide tube is configured to be outwardly expandable in the radial direction beyond the outer surface of the catheter body with the distal end in proximity to the inside wall of the target vessel. The catheter can include at least one sharpened needle comprising a needle fluid injection lumen in fluid communication with the catheter fluid injection lumen. In some embodiments, a portion of the at least one sharpened needle is located coaxially inside of the at least one guide tube. The catheter can include a proximal handle comprising an injection port in fluid communication with the catheter fluid injection lumen, the injection port comprising a check valve, the proximal handle configured to advance and retract the at least one guide tube and the at least one sharpened needle.

In some embodiments, the catheter can include three guide tubes and three sharpened needles. In some embodiments, the at least one sharpened needle is hollow and comprises a fluid egress near the distal end of the at least one sharpened needle and the catheter fluid injection lumen in fluid communication with the fluid egress of the at least one sharpened needle. In some embodiments, the proximal handle includes at least one indicia associated with the state of the catheter selected from the group consisting of: the position of the movement mechanism where the at least one guide tube and at least one injector tubes are both retracted, the position of the movement mechanism where the at least one guide tube is advanced but the at least one injector tube is retracted, and the position of the movement mechanism where the at least one guide tube and at least one injector tube are both advanced. In some embodiments, the fluid is ablative fluid and where the check valve is integrated with the proximal handle and configured to prevent the ablative fluid from flowing back out of the injection port. In some embodiments, the check valve is integrated with the proximal handle and configured to prevent air from entering the catheter fluid injection lumen. In some embodiments, the check valve is integrated with the proximal handle and configured to prevent blood from flowing back through the catheter.

In some embodiments, a catheter for fluid delivery through at least two injection needles into tissue outside of the interior wall of a target vessel of a human body is provided. The catheter can include a catheter body comprising an outer surface, a central axis extending in a longitudinal direction, and a fluid injection lumen. The catheter can include at least two guide tubes configured to advance distally and expand outwardly toward the interior wall of the target vessel. The catheter can include a support structure. The catheter can include at least two injector tubes with distal injection needles. In some embodiments, each of the distal injection needles comprises an injection lumen in fluid communication with the fluid injection lumen of the catheter body. In some embodiments, the at least two injector tubes with distal injection needles are configured to be advanced outwardly, guided by the at least two guide tubes to penetrate the interior wall of the target vessel. In some embodiments, the injection needles comprise a distal opening for fluid delivery into the tissue outside of the interior wall of the target vessel. The catheter can include a check valve positioned near the proximal end of the fluid injection lumen.

In some embodiments, the support structure comprises a deflection surface, the deflection surface configured to deflect the distally moving guide tubes outward to a pre-set radial distance from the outer surface of the distal portion of the catheter body. In some embodiments, the catheter comprises three guide tubes. In some embodiments, the at least one distal injection needle is hollow and includes fluid egress near the distal end of the injection needle and the fluid injection lumen of the catheter body is in fluid communication with the fluid egress of the at least one injection needle. In some embodiments, the check valve is located at the proximal end of the catheter body. In some embodiments, the catheter body comprises an injection port at the proximal end of the fluid injection lumen and the check valve is attached to a proximal end of the injection port. In some embodiments, the check valve is integral to the proximal portion of the fluid injection lumen.

In some embodiments, a catheter is provided. The catheter can include a catheter body comprising a fluid injection lumen. The catheter can include at least one guide tube comprising a distal end. In some embodiments, the at least one guide tube is moveable between a first position within the catheter body and a second position inclined away from the catheter body. In some embodiments, the at least one guide tube is configured to be positioned with the distal end in proximity to an inside wall of a target vessel. The catheter can include at least one penetrator comprising an injection lumen in fluid communication with the fluid injection lumen of the catheter body. In some embodiments, the at least one penetrator is configured to penetrate the inside wall of the target vessel. In some embodiments, a portion of the at least one penetrator located coaxially inside of the at least one guide tube. The catheter can include a proximal handle configured to advance and retract the at least one guide tube and the at least one penetrator. The catheter can include a check valve in fluid communication with the fluid injection lumen of the catheter body.

In some embodiments, the check valve is integrated into the proximal handle. In some embodiments, the check valve is integrated into a fluid injection port in a proximal portion of the catheter. In some embodiments, the fluid injection port includes a non-Luer connector. In some embodiments, the catheter further includes a vial of ablative fluid and at least one syringe. In some embodiments, the check valve is configured to allow ablative fluid to flow in one direction from a proximal injection port to an egress of the at least one penetrator and prevent ablative fluid from flowing in the opposite direction out of the proximal injection port.

In some embodiments, a catheter for fluid delivery through at least two injection needles into tissue outside of the interior wall of a target vessel of a human body is provided. The catheter can include a catheter body comprising an outer surface, a central axis extending in a longitudinal direction, and three concentric tubular structures comprising an outer tube, a middle tube, and an inner tube. In some embodiments, the inner tube structure comprises a fluid injection lumen. The catheter can include at least two guide tubes configured to advance distally and expand outwardly toward the interior wall of the target vessel. The catheter can include a support structure. The catheter can include at least two injector tubes with distal injection needles. In some embodiments, each of the distal injection needles comprises an injection lumen in fluid communication with the fluid injection lumen of the catheter body. In some embodiments, the at least two injector tubes with distal injection needles are configured to be advanced outwardly, guided by the at least two guide tubes to penetrate the interior wall of the target vessel In some embodiments, each of the distal injection needles comprises a distal opening for fluid delivery into the tissue outside of the interior wall of the target vessel. The catheter can include a rod positioned within the inner tube, the rod configured to reduce the volume within the inner tube by at least 50%.

In some embodiments, the support structure includes a deflection surface, the deflection surface configured to deflect the distally moving guide tubes radially outward from the outer surface of the distal portion of the catheter body. In some embodiments, the catheter comprises three guide tubes. In some embodiments, each of the distal injection needles is hollow and the fluid injection lumen in fluid communication with the distal openings of the injection needles. In some embodiments, the catheter can include a wire placed in each distal injection needle to reduce the volume within the distal injection needle. In some embodiments, the wire is radiopaque. In some embodiments, the wires comprises a material selected from the group consisting of: gold, platinum, tantalum, iridium, and tungsten filled urethane plastic. In some embodiments, the rod comprises a circular cross section. In some embodiments, the rod is hollow.

In some embodiments, a catheter for fluid delivery into a tissue outside of an interior wall of a target vessel of a human body is provided. The catheter can include a catheter body comprising an inner tube comprising an inner tube diameter, the inner tube comprising a fluid injection lumen. The catheter can include at least one needle guiding element configured to expand outwardly toward the interior wall of the target vessel. The catheter can include at least one injector tube comprising a distal sharpened needle and an injector tube lumen. In some embodiments, the at least one injector tube is in fluid communication with the fluid injection lumen of the catheter body. In some embodiments, the at least one injector tube is configured to be advanced outwardly, guided by the at least one needle guiding element. In some embodiments, each distal sharpened needle comprising a distal opening for fluid delivery into the tissue outside of the interior wall of the target vessel. The catheter can include a rod located inside the inner tube, the rod comprising a diameter of at least half of the inner tube diameter.

In some embodiments, the inner tube diameter is less than 0.5 mm. In some embodiments, the diameter of the rod is at least 0.25 mm. In some embodiments, the diameter of the rod is at least 75% of the inner tube diameter. In some embodiments, the diameter of the rod is configured to reduce dead space within the catheter body. In some embodiments, the rod is solid. In some embodiments, the rod is hollow and comprises at least one closed end. In some embodiments, the rod comprises a plastic material.

In some embodiments, a catheter for fluid delivery into a tissue outside of an interior wall of a target vessel of a human body is provided. The catheter can include a catheter body comprising a central axis extending in a longitudinal direction and a fluid injection lumen. The catheter can include at least two needle guiding elements configured to expand outwardly toward the interior wall of the target vessel. The catheter can include at least two injection tubes. In some embodiments, each injection tube comprises an injector tube lumen and a distal sharpened needle. In some embodiments, each injection tube lumen is in fluid communication with the fluid injection lumen of the catheter body. In some embodiments, the at least two injection tubes are configured to be advanced outwardly, guided by the at least two needle guiding elements. In some embodiments, each distal sharpened needle comprises a distal opening for fluid delivery. The catheter can include a rod within the fluid injection lumen. In some embodiments, the rod can run along a proximal section and a central section of the catheter. In some embodiments, the rod is configured to reduce an internal volume of the fluid injection lumen the catheter.

In some embodiments, the rod is flexible. In some embodiments, a wire is within the injection tube to reduce an internal volume within the injector tube lumen.

In some embodiments, a catheter for fluid delivery to a volume of tissue in outside of the inside wall of a target vessel in a human body is provided. The catheter can include a catheter body comprising an outer surface and a central axis extending in a longitudinal direction. The catheter can include at least one guide tube comprising a distal end. In some embodiments, the at least one guide tube is configured to be outwardly expandable a radial distance of at least 0.5 millimeter beyond the outer surface of the catheter body with the distal end in proximity to the inside wall of the target vessel. The catheter can include at least one sharpened needle comprising an injection lumen with distal injection egress. In some embodiments, a portion of the at least one sharpened needle located coaxially inside of the at least one guide tube. The catheter can include a proximal handle configured to advance and retract the at least one guide tube and the at least one sharpened needle. In some embodiments, the proximal handle comprises an unlock mechanism comprising a locked state and an unlocked state. In some embodiments, the proximal handle comprises a movement mechanism configured to allow the relative longitudinal movement of the at least one guide tube with respect to the catheter body and the at least one sharpened needle with respect to the at least one guide tube. In some embodiments, the proximal handle comprises a radial adjustment mechanism to adjust the radial distance to which the at least one guide tube is outwardly expandable.

In some embodiments, the catheter can include three guide tubes and three sharpened needles. In some embodiments, the at least one sharpened needle is hollow and includes fluid egress near the distal end of the at least one sharpened needle and the catheter comprises an injection lumen in fluid communication with the fluid egress of the at least one sharpened needle. In some embodiments, the at least one sharpened needle has a distal end that forms an electrode, the catheter body comprising a wire that runs the length of the catheter to conduct electrical signals between the at least one electrode and a connecting means near the proximal end of the catheter, the conducting means configured to connect the wire to external equipment. In some embodiments, the external equipment includes electronic systems selected from the group consisting of: means to measure electrical signals, means to measure electrical signals sensed by the electrode of the at least one sharpened needle, means to provide electrical stimulation signals to the electrode of the at least one sharpened needle, and means to provide energy based ablation through the electrode of the at least one sharpened needle. In some embodiments, the radial adjustment mechanism to adjust the radial distance to which the at least one guide tube is outwardly expandable has at least two pre-set selectable settings. In some embodiments, there are 3 or more pre-set selectable settings. In some embodiments, the radial adjustment mechanism is a slider. In some embodiments, the proximal handle comprises at least one indicia associated with the state of the catheter selected from the group consisting of: the position of the movement mechanism where the at least one guide tube and at least one sharpened needle are both retracted, the position of the movement mechanism where the at least one guide tube is advanced but the at least one sharpened needle is retracted, the position of the movement mechanism where the at least one guide tube and at least one sharpened needle are both advanced, and the position of the radial adjustment mechanism to adjust the radial distance to which the at least one guide tube is outwardly expandable. In some embodiments, the proximal handle includes a graphic icon associated with the at least one indicia. In some embodiments, the at least one indicia associated with the position of the radial adjustment mechanism to adjust the radial distance to which the at least one guide tube is outwardly expandable is selected from the group consisting of: a text label, a circular icon, and a text label and a circular icon.

In some embodiments, a catheter for fluid delivery through at least two injection needles into tissue outside of the interior wall of a target vessel of a human body is provided. The catheter can include a catheter body comprising an outer surface, a central axis extending in a longitudinal direction, and a fluid injection lumen. The catheter can include at least two guide tubes configured to advance distally and outwardly toward the interior wall of the target vessel.

The catheter can include at least two injector tubes with distal injection needles comprising an injection lumen in fluid communication with the fluid injection lumen of the catheter body. In some embodiments, the at least two injector tubes with distal injection needles are configured to be advanced outwardly, guided by the at least two guide tubes to penetrate the interior wall of the target vessel. In some embodiments, each of the distal injection needles comprises a distal opening for fluid delivery into the tissue outside of the interior wall of the target vessel. The catheter can include a mechanism for adjusting the pre-set radial distance from the outer surface of the distal portion of the catheter body to which the at least two guide tubes move outward.

In some embodiments, the catheter can include three guide tubes. In some embodiments, the at least one injector tube with distal injection needle is hollow and includes fluid egress near the distal end of the injection needle and the catheter includes an injection lumen in fluid communication with the fluid egress of the at least one injection needle. In some embodiments, the mechanism to adjust the pre-set radial distance to which the at least one guide tube is outwardly expandable has at least two pre-set selectable settings. In some embodiments, there are 3 or more pre-set selectable settings. In some embodiments, the mechanism is a slider.

In some embodiments, a method for delivery of a fluid outside of the inside wall of a target vessel of a human body is provided. The method can include advancing into the target vessel a catheter. The catheter can include a catheter body, a fluid injection lumen, at least one guide tube comprising a distal end, and at least one injector tube with distal needle located coaxially within the at least one guide tube. In some embodiments, the at least one guide tube is extendable in the radial direction away from the catheter body. In some embodiments, the at least one injector tube is extendable beyond the distal end of at least one guide tube. In some embodiments, the distal needle of the at least one injector tube comprises fluid egress in fluid communication with the fluid injection lumen. The catheter can include a proximal handle comprising a longitudinal movement mechanism to advance and retract the at least one guide tube and at least one injector tube and a radial adjustment mechanism to adjust a radial distance to which the at least one guide tube is outwardly expandable with respect to the catheter body. The method can include determining the diameter of the vessel to be treated. The method can include using the radial adjustment mechanism to adjust the radial distance to which the at least one guide tube is outwardly expandable with respect to the catheter body to select an appropriate radial distance for the diameter of the vessel. The method can include operating the longitudinal movement mechanism to advance the at least one guide tube away from the catheter body to the radial distance determined by the radial adjustment mechanism, the distal end of the at least one guide tube is in proximity to the inside wall of the vessel. The method can include operating the longitudinal movement mechanism to extend the at least one injector tube a preset distance beyond the distal end of at least one guide tube, causing the at least one injector tube to penetrate through the inside wall of the target vessel placing the fluid egress of the at least one needle into a volume of tissue outside of the inside wall of the target vessel. The method can include attaching a fluid source to the catheter. The method can include injecting fluid through the fluid injection lumen and out of the fluid egress into a volume of tissue outside of the inside wall of the vessel.

In some embodiments, the catheter includes three guide tubes and three injector tubes with distal needles. In some embodiments, the radial adjustment mechanism to adjust the radial distance to which the at least one guide tube is outwardly expandable with respect to the catheter body has at least two preset selectable settings.

### SUMMARY

The use of guide tubes as needle guiding elements of the catheters, such as the Peri-vascular Tissue Ablation Catheter (PTAC) of U.S. Pat. No. 9,179,962, are disclosed in the “Fischell Patents” that include Fischell et al. U.S. Pat. No. 9,056,185, issued Jun. 16, 2015 U.S. Pat. No. 9,179,962, issued Nov. 10, 2015, U.S. Pat. No. 9,254,360, issued Feb. 9, 2016, U.S. Pat. No. 9,301,795, issued Apr. 5, 2016, U.S. Pat. No. 9,320,850, issued Apr. 26, 2016, U.S. Pat. No. 9,526,827, issued Dec. 27, 2016, U.S. Pat. No. 9,539,047, issued Jan. 10, 2017, and U.S. Pat. No. 9,554,849, issued Jun. 3, 2014, which are hereby incorporated by reference in their entirety. Such guiding elements can be important, in certain embodiments, for the support of small diameter needles to access the volume of tissue deep to the inside wall of a target vessel.

The present disclosure is a Peri-vascular Fluid Injection Catheter (PFIC) with a number of embodiments that improve upon the prior art.

While certain catheters include a means to adjust the depth of needle penetration beyond the end of the guide tube/needle guiding element, they lack the description of means to adjust the length of the guide tubes to allow use of the catheter in vessels of widely different inside diameter. This is important as some patients requiring a renal denervation may have one or more smaller renal arteries; may have an accessory artery; or may have a very short main renal that bifurcates into two smaller arteries instead of one large one which would remove them as a candidate for the procedure that can provide them significant benefit or make it necessary for them to undergo RF ablation which is more painful and a longer procedure using the Simplicity catheter from Medtronic.

One embodiment of the PFIC includes means to allow the PFIC to operate in a wider range of vessel diameters than the other catheters described in the prior art. Specifically, the PFIC includes two approaches to doing this.
1. In a first approach, the Guide Tubes are shortened and may include an angulated distal end to provide a small vessel version of the PFIC,
2. An embodiment of the PFIC can have means to adjust the extension of the needle guiding elements/guide tubes beyond the openings in the distal portion of the PFIC. This can be done in combination with the ability to adjust needle extension beyond the end of the guide tubes or with a fixed length needle extension beyond the end of the guide tube. The adjustable needle extension has merit for applications like injection at different depths into a malignancy. For use in Renal Denervation or other blood vessel peri-vascular injection applications, the arterial wall may not differ much for smaller vs. larger blood vessels and therefore a fixed needle extension beyond the end of the guide tube is suitable while the feature of adjustable guide tube extension is highly desirable.

One embodiment of embodiment 2 includes a handle with the capability to adjust the relative starting position of the catheter outer body to the middle tube that controls the longitudinal position of the guide tubes. This adjustment could be continuous or have two to 5 preset steps. Icons, a scale or label on the handle can indicate the diameter or range of diameters of vessels that could be treated for each present or continuous adjustment mechanism. An embodiment for Renal Denervation can have two preset positions of a mechanism such as a slider that snaps into each position. For example, one preset can cover renal artery inside diameters of 2-4 mm, the other can cover a range of 4-7 mm. It is also envisioned that the unconstrained diameter of the guide tubes can be slightly larger than the largest diameter for which the preset is labeled. For example a 5 mm unconstrained diameter for the guide tubes expansion in air for the 2-4 mm range and an 8 mm diameter for the guide tube for the 4-7 mm range.

It is also envisioned that rather than moving the start position for the middle tube longitudinally with respect to the handle that is fixedly attached to the main catheter outer tube/body, one could have the adjustment move the main catheter outer tube/body with respect to the handle. The guide tube and needle extension and retraction mechanism can have any feature described herein or in any of the patents incorporated by reference.

The radius of curvature matched between the Guide tubes and needles is described herein and in the Fischell Patents, incorporated by reference. In some embodiments, it is also important that the needle radius of curvature be between 0.075″ and 0.125″ with 0.11″ as a radius so that the needle penetration into the vessel wall will not curve back too far and will have the fluid egress in the distal portion of the needle properly access the peri-vascular space.

The central axis of the end of the guide tubes should be approximately 90 degrees to the plan of the wall of the vessel when the outward moving guide tube engages the wall at the largest diameter vessel for a specific catheter design. Another way to describe this is that the plane of the distal tip of the guide tube should be parallel to the plane of the inside vessel wall to minimize trauma when the guide tube distal end presses against the inside wall. To achieve this an angulated distal end of the guide tube is envisioned so that in air at maximum deployed diameter, the guide tube should have its axis curved back greater than 90 degrees.

At the minimum diameter vessel for a specific design, the plane of the distal tip of the guide tube should be no less than 45 degrees off from being parallel to the plan of the inside wall of the vessel.

If rather than an adjustable catheter able to adapt to both small and large vessels, the there is a small vessel embodiment of the PFIC and at the upper range of vessel diameter the radius of curvature of the guide tubes does not allow the axis of the guide tube to reach 90 degrees when it engages the wall of the vessel, one feature of the small vessel PFIC is to chamfer the end of the guide tube, so that it is parallel to the wall of the vessel at the desired maximum diameter for use.

Another embodiment radial access PFIC has an overall increased length and reduced diameter to best allow insertion of the radial PFIC from radial artery which is known to reduce bleeding complications. It is also envisioned that even without a reduced diameter PFIC, use of a long sheath or sheathless guiding catheter can facilitate insertion of a 7 french PFIC through a hole in the artery equivalent to that of a 6 french sheath.

The ideal range of length from the distal end of the handle to the guide tube exit openings should be 80 cm to 100 cm for femoral access increasing to 115 cm to 130 cm for radial access through a 100 cm radial access guiding catheter.

The use of 3 hypotubes can make the majority of the length of the catheter too stiff to be placed in anything but a very long package that may be less desirable than a catheter that can be wound into a spiral package or placed in a shorter package. The flexible PFIC includes several embodiments that can facilitate a PFIC design that can be packaged in a more easily stored box. These embodiments can include use of wire braid or helical wire plastic tubes for a portion or all of the inner, middle tube or outer tubes of the PTAC. It is also envisioned that if three metal tubes are used, laser cut holes or slots in the middle or outer tube could greatly increase flexibility without losing pushability needed to deploy the guide tubes and needles. An additional embodiment could place one or more flexible connectors in the central portion of the hypotubes to allow the PFIC to be bent back on itself for storage in a shorter package. These connectors can be plastic, NITINOL, or a wire braided or helical wire impregnated plastic tube. By having the flexible connectors being relatively short, little pushability will be lost compared to a that of an unconnected/unsegmented hypotube.

Some of the prior art embodiments describe catheters that can measure nerve activity but lack the capability to take other important physiological measurements that could be accomplished and would be of value once the catheter is placed into the target vessel. As a primary use of the PFIC is to provide renal denervation for the treatment of hypertension, an augmented embodiment of the PFIC is envisioned to include a pressure sensor on or in the body of the PFIC that can be used to determine if the denervation procedure has been effective in lowering blood pressure. Other sensors that could be of value include:

1. a flow wire for measuring blood flow into the kidney

2. a temperature sensor

3. an ultrasound imaging transducer and sensor

Another important feature of the PFIC is the use of an integrated check valve at the proximal end of the PFIC to ensure that ablative fluid injected into the injection lumen of the PFIC cannot flow back out of the proximal injection port And air cannot accidentally get introduced into the infusion lumen.

Prior art methods also describe the use of the prior art catheters with a first step of priming the injection lumen with saline. While priming is important to remove air from the injection lumen, priming with saline will dilute the ablative fluid that follows in its injection into peri-vascular tissue for the renal denervation application. The PFIC method of use envisions an improved method that simplifies the operation of the PFIC for renal denervation by having its method of use include the following steps:
1. remove the PFIC from its package onto the sterile field in the cath lab,
2. expand the guide tubes and needles if not already packaged in the expanded state,
3. remove the vial of ablative fluid from its package and draw sufficient fluid into a syringe to flush the injection lumen of the PFIC,
4. connect the syringe to the PFIC injection port at its proximal end and inject a sufficient amount of ablative fluid to flush the injection lumen with some small amount exiting the distal end of the injection needles,
5. flush the other lumens of the PFIC with saline and also use saline to rinse the distal portion of the PFIC to ensure no ablative fluid lingers on the outside of the catheter,
6. retract the needles and guide tubes back into the PFIC in preparation for use,
7. if the PFIC is has adjustable vessel size, set the appropriate diameter or diameter range using the mechanism in the PFIC handle,
8. insert the distal portion of the PFIC through a guiding catheter or sheath into a first renal artery of the patient using radial or femoral access methods,
9. expand the guide tubes to center the PFIC distal portion in the renal artery,
10. advance the injector tubes with distal needles through the guide tubes,
11. withdraw the appropriate predetermined amount of ablative fluid from the vial into the syringe and attach the syringe to the injection port of the PFIC,
12. inject the proper amount of ablative fluid into the injection lumen of the PFIC and as the lumen is already filled with ablative fluid the amount injected from the syringe will also egress from the distal egress of the injection needles,
13. retract the injector tubes with distal needles then retract the guide tubes,
14. withdraw the PFIC back into the guiding catheter,
15. if a second artery is to be treated on the same side, adjust the range of vessels diameter associated with the second artery and repeat steps 9 through 14,
16. if no second artery, then relocate the guiding catheter or sheath to allow access to the renal artery to the patient's other kidney and repeat steps 9 through 15, the PFIC may be removed from the guiding catheter or sheath during relocation to re-prime the infusion lumen and verify that all three needles are patent,
17. remove the PFIC from the body.

This method is also applicable to any renal denervation catheters described herein or any catheter described in patents incorporated by reference. An additional feature is to reduce the internal volume of the injection lumens of the catheter by insertion of a rod within the primary injection lumen of the PFIC, which runs along the proximal and central sections of the catheter.

This method is also applicable if the PFIC only functions for a single range of vessel sizes with the difference being that one would first treat all of the vessels on one side using two sizes of catheters if needed, then move the guiding catheter or sheath then treat the other side with two sizes if needed.

Fischell et al. in U.S. Pat. No. 10,485,951, issued Nov. 26, 2019 and incorporated by reference herein, describe techniques for packaging a fluid injection catheter with needles and guide tubes/needle guiding elements. Kits described herein can include items important for use in a renal denervation procedure. Also it can be highly desirable for there to be additional “spare part” mini-kits of components that can assist in the treatment of multiple vessels or more complex situations. Specifically, one can envision a spare kit with a vial of ablative fluid and one or two syringes. This is even more important if the infusion port of the PFIC includes a non-standard connector as described in U.S. Pat. No. 9,320,850 which is incorporated by reference herein.

An additional part that could be included in a spare parts kit is the introducer used to facilitate placement of the fixed wire version of the PFIC into the proximal end of the guiding catheter or sheath used to deliver the catheter into the target vessel of the human body.

Another potential feature of the PFIC is that it is envisioned that the some or all of the plastic used to form the guide tubes may be formed from a plastic impregnated with radiopaque particles such as tungsten filled urethane. This may eliminate the need for radiopaque bands and reduce the cost of manufacturing the PFIC.

While certain catheters utilize a guide tube/needle guiding element through which an injector tube with distal injection needle is coaxially advanced, it is envisioned that an injection tube/needle with a proximal larger diameter section could provide sufficient support to allow reliable advancement, centering and support for the distal penetrating portion of the needle. It is also envisioned that a single expandable arm combining the guide tube and injector tube with distal needle could function to both center the PFIC and have an appropriate penetration of the inside wall of the target vessel by the injector tube. An advantage of a single arm structure is that is reduced the need for three tubes for the proximal and central portion of the PFIC allowing for a smaller outside diameter for the catheter.

Throughout this specification the terms injector tube with distal injection needle is used to specify a tube with a sharpened distal end that penetrates into tissue and is used to inject a fluid into that tissue. Such a structure may at times also be called a hypodermic needle, an injection needle or simply a needle. In addition, the terms element and structure may be used interchangeably within the scope of this application. The term Luer fitting may be used throughout this application to mean a tapered Luer fitting without a screw cap or a Luer Lock fitting that has a screw cap.

Thus it is an object to have a Peri-vascular Fluid Injection Catheter (PFIC) with a mechanism allowing the operator to adjust the device for use in 2 or more ranges of vessel size.

Another object of the PFIC is to have the adjustment for vessel size range incorporated in the handle of the PFIC.

Another object is to have a lengthened version of the PFIC suited for delivery to the desired site in the human body through the radial artery in the arm of a patient.

Still another object is to include a flexible rod or wire within the primary fluid injection lumen that runs along the proximal and central portions of the catheter.

Still another object of the PFIC is to have a check valve incorporated into the fluid injection port in the proximal portion of the PFIC.

Still another object of the PFIC is to include flexible coupling elements in one or more of the tubular structures in the main body of the catheter to facilitate packaging in a package smaller than the total length of the PFIC.

Still another object is to have a portion of the guide tubes formed from a radiopaque plastic material.

Yet another object of the method of use for the fluid injection catheters where the injection lumen is flushed with ablative fluid instead of saline during device pre-procedure preparation so as to reduce dilution of the ablative fluid that is delivered to the peri-vascular space.

Yet another object of the PFIC is to include auxiliary kits packaged separately from the PFIC, the kits including at least a vial of ablative fluid and a syringe.

Yet another object of the PFIC is to have one or more of the tubular elements in the main body of the catheter be constructed to be more flexible than a stainless steel hypotube.

Yet another object is to include additional sensors for blood pressure, blood flow rate, temperature and ultrasonic imaging.

Yet another object of the PFIC is to replace guide tubes and needles with a single extendable needle having a thickened diameter portion in its proximal portion.

Yet another object of the PFIC is to combine the guide tube and injector tube with distal needle in a single expandable arm.

Yet another object of the PFIC is to have curved injector tubes with distal needles having a radius of curvature between 0.05″ to 0.15″ with a radius of curvature being between 0.10″ and 0.12″ in some embodiments.

These and other features and advantages of this disclosure will become obvious to a person of ordinary skill in this art upon reading of the detailed description including the associated drawings and the claims.

### BRIEF DESCRIPTION OF THE DRAWINGS

FIG. 1 is a schematic view of a longitudinal cross-section of a distal portion of the PTAC in its open position for delivery of fluid into a volume of tissue outside of the inside wall of a target vessel.

FIG. 2 is a longitudinal cross-section of a distal portion of the PTAC in its open position for delivery of fluid into a volume of tissue outside of the inside wall of a target vessel.

FIG. 3 is a side view of an embodiment of the proximal handle.

FIG. 4 is a longitudinal cross section of the PFIC showing the addition of a rod in the primary injection lumen of the catheter designed to reduce the dead space within the injection lumen.

FIGS. 5A and 5B are side views of the embodiment of the proximal handle designed for use with the PFIC which includes the mechanism to adjust the range of vessel diameters that can be treated.

FIG. 6A is a cross sectional view of an embodiment of the distal portion of the guide tubes and injector tubes with distal needles in the expanded state.

FIG. 6B is a cross sectional view of an embodiment of the PFIC injector tube designed to work without a separate guide tube for deployment of the injection needle

FIG. 6C is a cross sectional view of an embodiment of the PFIC that integrates together the guide tube and injector tube into a single extendable arm.

FIG. 7A is a cross sectional view showing the engagement of the PTAC guide tube with the inside wall of a target vessel.

FIG. 7B is a cross sectional view showing the engagement of the PFIC with angulated guide tube distal end with the inside wall of a target vessel.

FIG. 8A is a longitudinal cross section of a central portion of the PTAC showing the three concentric hypotubes.

FIG. 8B is a longitudinal cross section of a central portion of an embodiment of PFIC showing the three concentric plastic tubes with an integral helically wound flat metal wire.

FIG. 8C is a longitudinal cross section of a central portion of an embodiment of the PFIC showing flexible connecting tubes between distal and proximal portions of the three concentric hypotubes.

FIG. 8D is a longitudinal cross section of a central portion of an embodiment of the PFIC showing flexible non-kinking connecting tubes with integral helically wound flat metal wire used to connect distal and proximal portions of three concentric hypotubes.

FIG. 9 is a schematic view showing the folded PFIC as it might be positioned for packaging.

### DETAILED DESCRIPTION

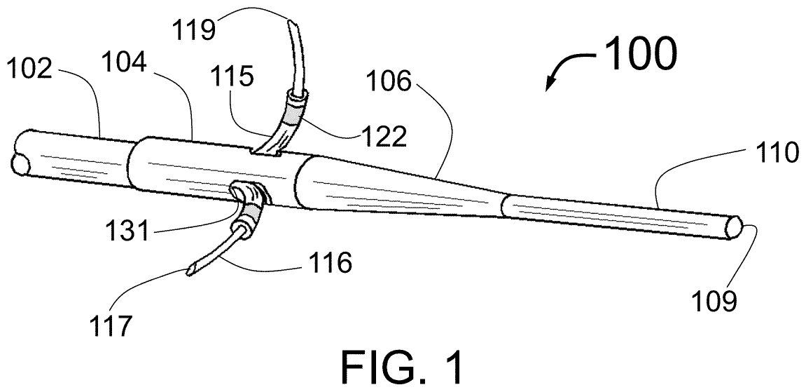

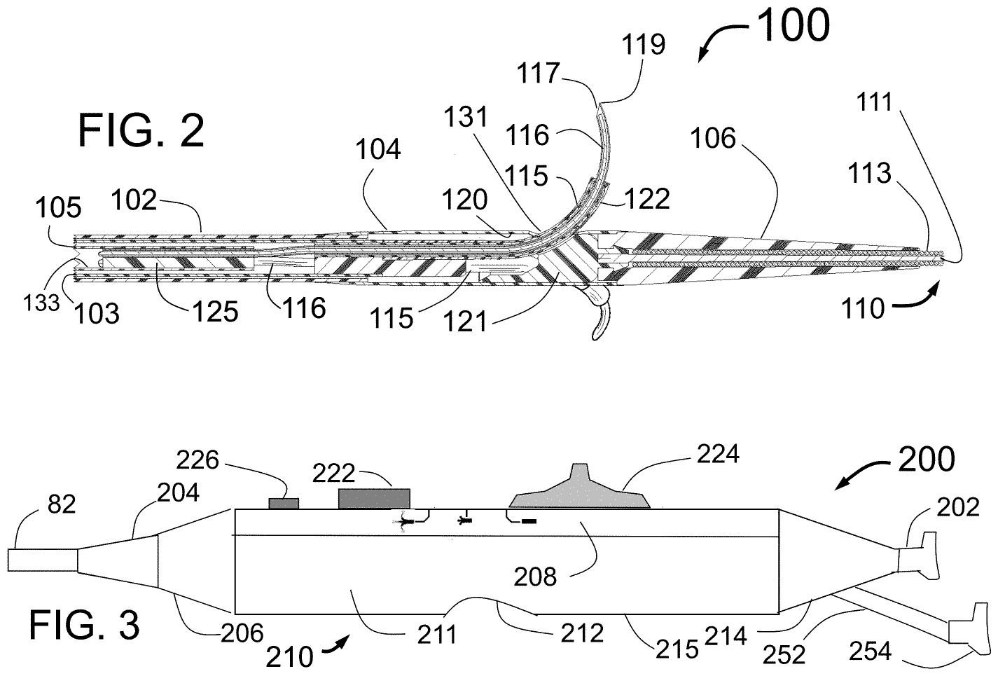

FIG. 1 is a schematic view of a longitudinal cross-section of a distal portion of the PTAC 100. Certain embodiments and features of the PTAC are disclosed and shown in FIG. 2 of Fischell et al. U.S. Pat. Nos. 9,179,962, 9,254,360, 9,301,795, 9320,850, 9,526,827, 9,539,047, and 9,554,849, incorporated herein by reference. The PTAC 100 is shown in its open position for delivery of fluid into a volume of tissue outside of the inside wall of a target vessel.

The PTAC 100 includes an outer tube 102, outer tube extension 104 having distal openings 131 through which the guide tubes 115 with radiopaque markers 122 are advanced outward from the outer tube extension 104 of the PTAC 100. Also shown is the tapered section 106 and fixed guide wire 110 with distal tip 109. The injector tubes 116 with distal injection needles 119 and needle distal openings 117 providing fluid egress are shown in their fully deployed positions. The PTAC 100 has three guide tubes with the third tube hidden behind the catheter and not visible in this schematic view. Although the PTAC 100 has three guide tubes 115, it is envisioned that other embodiments could have as few as one or as many as eight guide tubes with an optimum number being three or four. A larger diameter target vessel might suggest the use of as many as 4 to 8 guide tubes 115 with coaxial injector tubes 116.

Different shapes are envisioned for the distal openings (or windows) 131 in the outer tube extension 104 where the guide tubes 115 exit. These possible shapes include a racetrack design with curved (e.g., round) proximal and distal ends and straight sides in the axial direction, oval or round shapes. It is also envisioned that there could be a movable flap covering the opening 131 or a slit that could be opened to make the outer surface of the PTAC smooth for better delivery into the renal artery.

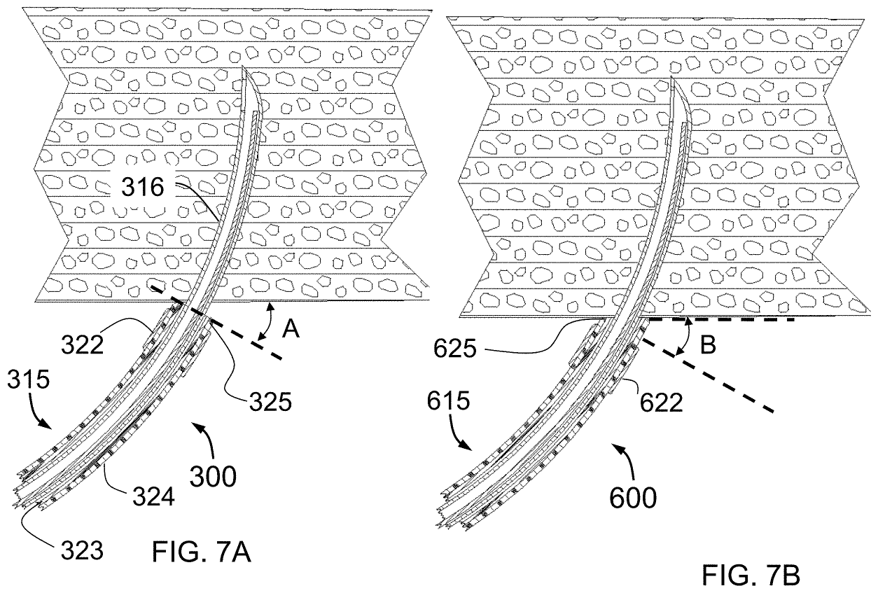

An important feature of this catheter is the guide tubes 115 that act as needle guiding elements for the ultra-thin injector tubes 116 with distal injection needles 119. While not shown in FIG. 1 but shown in FIG. 6A, an embodiment can include non-coring needles.

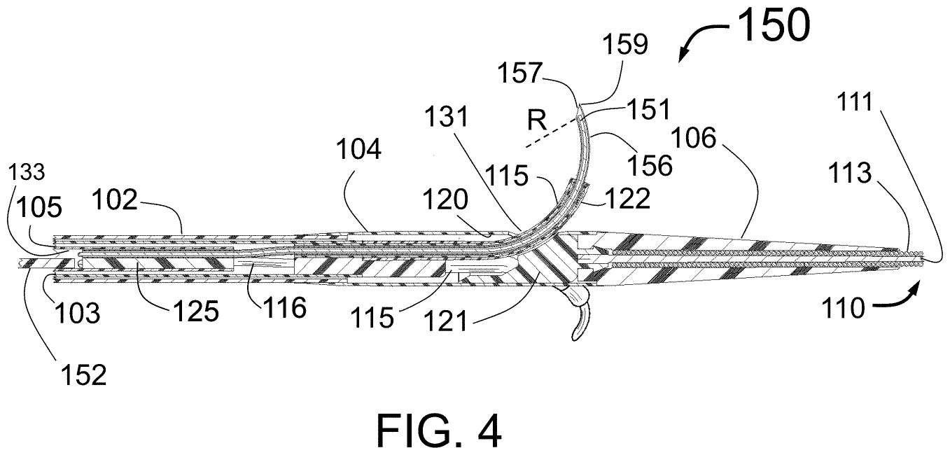

FIG. 2 is a longitudinal cross-section of the distal portion of the Peri-vascular Tissue Ablation Catheter PTAC 100. Certain embodiments and features of the PTAC are disclosed and shown in FIG. 3 of Fischell et al. U.S. Pat. Nos. 9,179,962, 9,254,360, 9,301,795, 9320,850, 9,526,827, 9,539,047, and 9,554,849, incorporated herein by reference. The proximal end of FIG. 1 shows the three concentric tubes, the outer tube 102, middle tube 103 and inner tube 105 which form the central portion of the PTAC 100. The outer tube 102 is attached to the outer tube extension 104 which is in turn attached to the tapered section 106. The fixed guide wire 110 with core wire 111 and outer layer 113 extends distally from the distal end of the tapered section 106. It should be noted that only part of the length of the guide wire 110 is shown in FIG. 1.

FIG. 2 shows the guide tube 115 with radiopaque marker 122 in its fully advanced position placed through the opening 131 in the outer tube extension 104. The interior surface of the outer tube extension 104 forms part of the tubular shaft 120 can be made from a stiff material such as a metal or high durometer plastic so that it will be relative rigid as the guide tubes 115 are advanced and retracted.

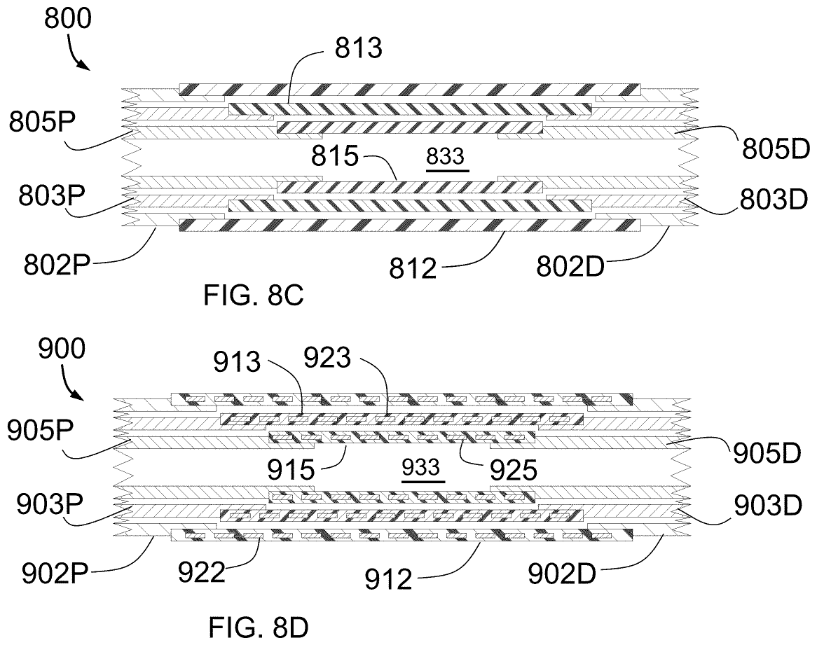

An embodiment of the PTAC 100 of the uses four different tubular structures instead of just an outer tube 102 and outer tube extension 104. Specifically, the proximal section of each of the three concentric tubes can be a metal hypotube. The metal hypotube can connect at its distal end to a relatively stiff plastic tube about 20 cm long that can in turn connect to a softer more flexible plastic tube about 10 cm long which can be the tube 102 shown in FIG. 1. FIG. 8A shows a longitudinal cross section of the hypotube section of the PTAC 100.

In an embodiment, the middle tube 103 attaches to, a proximal metal hypotube and the inner tube 105 can also attach to proximal portion formed from a metal hypotube.

The central buttress 121 shown in FIG. 2, supports the guide tube 115 both as it is pushed distally and after it is fully deployed. This central buttress 121 also provides radial support for the advanced guide tubes 115 that prevents the guide tubes 115 from backing away from the interior wall of the target vessel as the injector tubes 116 with sharpened needles 119 are advanced through the guide tubes 115 forward into and through the inner/interior wall of the target vessel to their desired position 2-5 mm beyond the inner wall of the target vessel. In exceptional cases, the injection needles 119 at the distal ends of the injector tubes 116 might be advanced as deep as 8 mm beyond the inner wall of the target vessel. Additional lateral support for the guide tubes 115 is provided by the sides of the openings 131 that in combination with the central buttress 121 are key to the radial and circumferential/lateral support both during guide tube 115 advancement, and as backup during delivery of the injection needles 119 through the interior wall of the target vessel. The buttress may comprise a deflection surface such as a curved or linear ramp, which may in a curved embodiment correspond to the radius of curvature of the distal surface of the guide tube 115.

The inner tube 105 with fluid injection lumen 133 connects through the manifold 125 to the three injector tubes 116, thus the lumens of the injector tubes 116 are in fluid communication with the fluid injection lumen 133. The inner tube 105 and manifold 125 can slide along the longitudinal axis of the PTAC 100 inside of the middle tube 103 which is shown with uniform diameter over its length including the portion coaxially outside of the manifold 125.

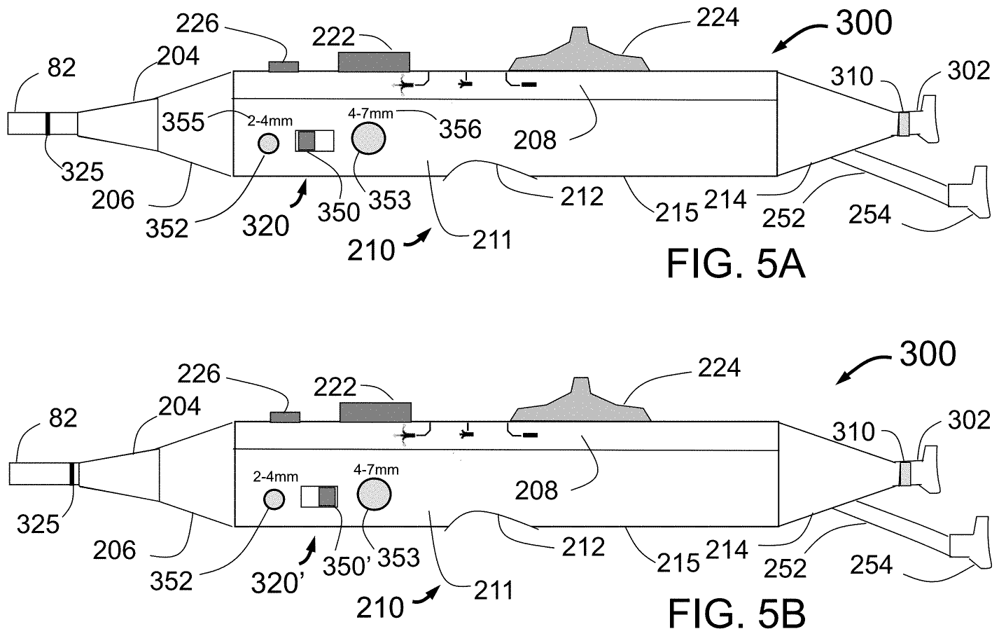

FIG. 3 is a side view of the control handle 200. Certain embodiments and features of the handle are disclosed and described in Fischell et al. U.S. patent application Ser. No. 16/039,234, filed Jul. 18, 2018, incorporated by reference herein, designed for use with the PTAC 100 of FIGS. 1 and 2 or the PFIC embodiments shown in FIGS. 4 through 9. The handle 200 is designed to simplify the operation of the catheter while including appropriate failsafe features.

The main body 210 of the handle 200 is of relatively rectangular or rounded cross section with beveled or rounded edges where the side surface of the handle 211 meets the bottom of the handle 215. A finger detent 212 improved the comfort of holding the handle 200 and is positioned so that the operators hand is situated to be able to best operate the primary controls of the handle including the unlock button 222, the unlock release button 226 and the slider 224. The slider 224 is an example of a longitudinal movement mechanism that can advance and retract the PTAC 100 guide tubes 115 of FIGS. 1 and 2 with respect to the PTAC 100 catheter body and can also advance and retract the PTAC 100 injector tubes 116 with needles 119 with respect to the guide tubes 115.

The unlock button 222 has locked (up) and unlocked (down) states. When depressed and released the unlock button 222 will stay in the unlocked (down) state and will allow longitudinal motion of the slider 224. If the operator depresses the unlock button 222 in error and wishes to pop it back up returning it to the locked (up) state, this can be accomplished by depressing the unlock release button 226.

The upper side of the handle 200 includes a rounded or beveled surface 208. A relock button 226 is also placed on the top of the handle 200.

Distal to the main body 210 is a tapered section 206, and distal to that is a strain relief 204 which is outside of the outer hypotube 102 seen for the PTAC 100 of FIG. 8A.

Proximal to the main body 210 is the proximal tapered section 214. Proximal to the proximal tapered section 214 is a connector 202 for attaching a syringe (not shown) or other fluid dispensing mechanism. The connector 202 may be a standard Luer or Luer lock connector or it may be a non-standard connector. The lumen of the connector 202 is in fluid communication with the lumen 133 of the inner tube 105 of the PTAC 100 of FIG. 2. A flushing tube 252 with Luer connector 254 is in fluid communication with two spaces: 1) the space between the inner tube 105 and middle tube 103 of FIG. 2 and 2) the space between the middle tube 103 and outer tube 102 shown in FIG. 2 and used to flush the catheter with saline to remove any air that might get into the patient's blood vessels before operation of the PTAC 100.

FIG. 4 shows a longitudinal cross sectional view of the PFIC 150. The PFIC 150 is a modified version of the PTAC of FIG. 2 with the changes including a specification of radius of curvature R for the injector tube 156 with distal needle 159, radiopaque wire 151 and fluid egress 157 replacing the injector tube 116 of FIG. 2 and the addition of an inserted flexible rod 152 inside of the lumen 133 of the inner tube 105. The radius of curvature R of the injector tube 156 with distal needle 119 can be between 0.05″ and 0.15″ so that the needles will have sufficient curve to match the curvature of the guide tube 115 but not curve back so much the fluid egress 157 does not properly in the peri-vascular space. The radius of curvature can be between 0.10″ and 0.12″.

The purpose of the rod 152 that can extend into the proximal portion of the PFIC 150 is to take up volume within the lumen 133 and the lumens 93, 733, 833 and 933 of the inner hypotubes 82, 702, 802 and 902 shown in FIGS. 8A though 8D to reduce the dead space in the primary injection lumen of the PFIC 150 that runs along the proximal and central sections of the catheter. It is envisioned that the rod 152 can be highly flexible so as not to affect the overall flexibility of the PFIC 150 proximal and central portions. While shown here as a solid rod, a hollow tube with closed ends could be used to increase flexibility. Ideally whether solid or tubular, the rod 152 can be formed from a low durometer plastic, that may be for example, Urethane, Tecothane or Pebax. The other element numbers of FIG. 4 are unchanged from those in the detailed description associated with FIG. 2 of this specification.

The catheter can include an outer surface. The catheter can include a central axis extending in a longitudinal direction and three concentric tubular structures, an outer tube, a middle tube and an inner tube. The inner tube structure can have a fluid injection lumen. The catheter can include at least two guide tubes adapted to advance distally and expand outwardly toward the interior wall of the target vessel. The catheter can include a mechanical support structure including a deflection surface, the deflection surface deflecting the distally moving guide tubes radially outward from the outer surface of the distal portion of the catheter body. The catheter can include at least two injector tubes with distal injection needles each having an injection lumen in fluid communication with the fluid injection lumen of the catheter body. The at least two injector tubes with distal injection needles are adapted to be advanced outwardly, guided by the at least two guide tubes to penetrate the interior wall of the target vessel. The injection needles can have a distal opening for fluid delivery into the tissue outside of the interior wall of the target vessel. The catheter can have three guide tubes. At least one needle can be hollow and includes fluid egress near the distal end of the needle, with the catheter having an injection lumen in fluid communication with the fluid egress of the at least one needle.

The catheter can include a rod positioned within the inner tube, the rod adapted to reduce the volume within the inner tube by at least 50%. The rod can reduce the volume within the inner tube by 50%, 55%, 60%, 65%, 70%, 75%, 80%, 85%, 90%, 95%, or any range of the forgoing values. The rod can reduce the volume of the fluid within the catheter including all fluid pathways by 50%, 55%, 60% 0, 65%, 70%, 75%, 80%, 85%, 90%, 95%, or any range of the forgoing values. The rod can minimize dead space within the catheter, which is advantageous when injecting ablative agents. The rod can reduce the dead space/internal volume in the catheter injection lumen. The rod can increase the resistance to flow which can beneficially slow down the rate of infusion.

Reducing the internal volume of the catheter minimizes the amount of saline needed to flush the ablative fluid out of the catheter into the desired volume of tissue. The dead space can be less than 0.5 ml and in some cases less than 0.2 ml. In some embodiments, dead space can be reduced to less than 0.1 ml. The inner tube can be a small diameter such as less than 0.5 mm inner diameter for fluid injection. The rod can be placed along the entire inner tube, or a portion thereof, to reduce the volume of the hypotube and thus reduce the catheter dead space. In some embodiments, the total internal volume or dead space from a proximal part to a distal end of an infusion flow path of the catheter is 0.5 ml, 0.4 ml, 0.3 ml, 0.2 ml, 0.1 ml, or any range of the foregoing values.

In some embodiments, the rod equalizes the flow rate between two or more injector tubes. In some embodiments, the rod equalizes the pressure between two or more injector tubes. In some embodiments, the rod equalizes the flow volume between two or more injector tubes. The rod can be centered within the catheter. The rod can be off-centered with the catheter. The rod can equally obstruct the flow for all flow paths to the needles. The rod can have a circular cross section. The rod can be other cross-sections including oval or polygonal. The rod can be hollow inside. The rod can be solid. The rod can be flexible. The rod can be radiopaque. The rod can be radio translucent.

The catheter can further include a wire placed in each injection needle to reduce the volume within the injection needle. The wire can be radiopaque. The wires can be formed from a material including gold, platinum, tantalum, iridium, and/or tungsten filled urethane plastic. In addition to providing better visibility, the radiopaque wire in the lumen of each injector tube reduces the internal volume or dead space within the injector tube. The rod can be separate from the wire. The rod and the wire can be separate materials. The rod and the wire can be the same material. In some embodiments, the rod and the wires are integrally formed.

Another potential feature of the PFIC 150 is that it is envisioned that the plastic used to form the guide tube 115 may be formed from a plastic impregnated with radiopaque particles such as tungsten filled urethane. This can eliminate the need for the radiopaque band 122.

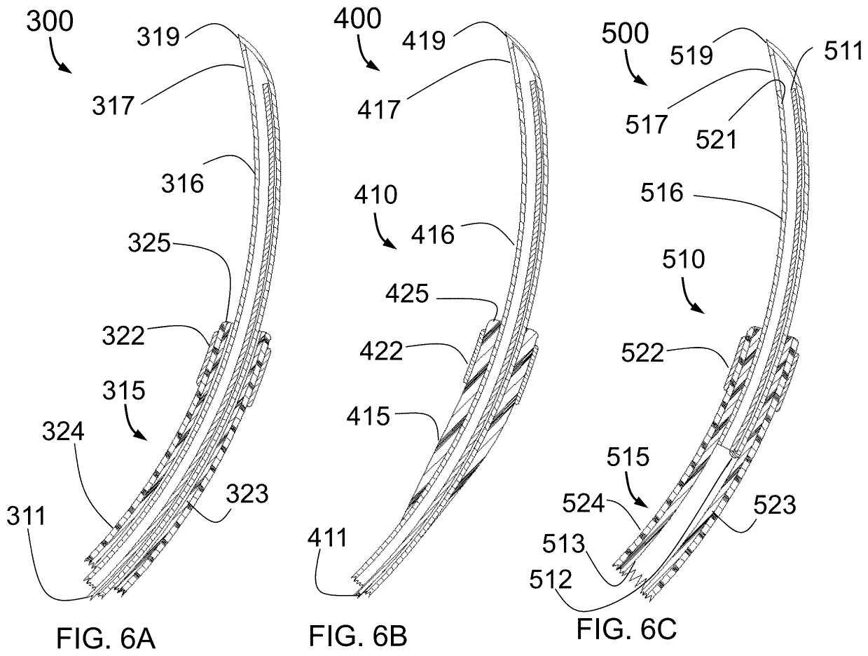

FIGS. 5A and 5B show the PFIC handle 300 which is a modification of the handle 200 of FIG. 3. The additions to the handle 200 include:
1. a check valve 310 integrated into the proximal connector 302 which is the fluid port for infusion of the fluid that will egress through the opening 117 in the injection needles 119 of FIG. 4. The check valve provides the advantage of preventing blood from flowing back through the primary infusion lumen 133 of FIG. 4,
2. a vessel size selection mechanism 320 including the slider 350 and vessel range markers 353 for larger vessels and 352 for smaller vessels. The markers 352 and 353 as shown in FIG. 5A include labels 355 and 356 respectively showing the vessel range where the larger vessel range marker is shown with a label 356 of 4-7 mm and the smaller vessel range marker with a label 355 of 2-4 mm. The slider 350 of FIG. 5A is shown in the position for the smaller range of vessels 352/355. The slider 350′ of the mechanism 320′ of FIG. 5B is shown in the position for the larger range of vessels 353/356.
3. an optional marker band 325 on the outer tube 82 which also indicates the relative longitudinal position of the outer tube 102 to the tapered sections 204 and 206 fixed to the outer case 211 of the handle 300.

The check valve 310 can be located in the proximal handle. The proximal handle can have an injection port in fluid communication with the catheter fluid injection lumen. The injection port can include the check valve 310. The proximal handle is further adapted to advance and retract the guide tubes and needles as described herein. In some embodiments, the check valve 310 can be located anywhere along the catheter fluid injection lumen. The check valve 310 can be located in fluid communication with any fluid pathway through the catheter. The catheter fluid injection lumen can be in fluid communication with the fluid egress of the at least one needle. The catheter fluid injection lumen can be in fluid communication with the fluid egresses of the at least three needles. The catheter fluid injection lumen can be in fluid communication with at least one hollow needle. The fluid egress can be ear the distal end of the needle. In some embodiments, the check valve 310 is positioned near the proximal end of the fluid injection lumen. In some embodiments, the check valve is located at the proximal end of the catheter body. The catheter body has an injection port at the proximal end of the fluid injection lumen and the check valve is attached to proximal end of the injection port. In some embodiments, the check valve is integral to the proximal portion of the fluid injection lumen.

In some embodiments, the at least one guide tube outwardly expandable a radial distance of at least 0.5 millimeter beyond an outer surface of the catheter body with the distal end in proximity to the inside wall of the target vessel. In some embodiments, the at least one guide tube outwardly expandable a radial distance of 0.5 millimeter, 0.6 millimeter, 0.7 millimeter, 0.8 millimeter, 0.9 millimeter, 1.0 millimeter, 1.1 millimeter, 1.2 millimeter, 1.3 millimeter, 1.4 millimeter, 1.5 millimeter, or any range of the foregoing valves. The proximal handle having a top surface, two side surfaces and a bottom surface is adapted to advance and retract the guide tubes and needles as described herein. The handle can include an unlock mechanism having a locked state and an unlocked state. The handle can include a movement mechanism designed to allow the relative longitudinal movement of the at least one guide tube with respect to the catheter body and the at least one needle with respect to the at least one guide tube. The handle including a mechanism to adjust the radial distance to which the at least one guide tube is outwardly expandable.

The at least one needle can include a fluid egress near the distal end of the needle. The at least one needle can have a distal end that forms an electrode. The catheter body further including a wire that runs the length of the catheter to conduct electrical signals between the at least one electrode and connecting means near the proximal end of the catheter. The conducting means adapted to connect the wire to external equipment. The external equipment includes electronic systems can include a means to measure electrical signals, a means to measure electrical signals sensed by the electrodes of the at least one needle, a means to provide electrical stimulation signals to the electrodes of the at least one needle, and/or a means to provide energy based ablation through the electrodes of the at least on needle.

The mechanism to adjust the radial distance to which the at least one guide tube is outwardly expandable has at least two pre-set selectable settings. In some embodiments, there are 3 or more pre-set selectable settings. In some embodiments, the mechanism is a slider. The slider can slide between two positions. The two positions can be labeled with words such as the diameter size and/or icons showing a larger expansion and a smaller expansion. The user can slide the mechanism to select between at least two pre-set settings for the radial distance. In some embodiments, the first pre-set setting is 0.5 millimeter, 1.0 millimeter, 1.5 millimeter, 2.0 millimeter, 2.5 millimeter, 3.0 millimeter, 3.5 millimeter, 4.0 millimeter, 4.5 millimeter, 5.0 millimeter, 5.5 millimeter, 6.0 millimeter, 6.5 millimeter, 7.0 millimeter, or any range of the foregoing valves.

In some embodiments, the second pre-set setting is 0.5 millimeter, 1.0 millimeter, 1.5 millimeter, 2.0 millimeter, 2.5 millimeter, 3.0 millimeter, 3.5 millimeter, 4.0 millimeter, 4.5 millimeter, 5.0 millimeter, 5.5 millimeter, 6.0 millimeter, 6.5 millimeter, 7.0 millimeter, or any range of the foregoing valves, or any range of the foregoing valves. In some embodiments, the first pre-set setting is between 2 millimeter and 4 millimeter. In some embodiments, the second pre-set setting is between 4 millimeter and 7 millimeter.

The proximal handle can include at least one label associated with the state of the catheter. The label can indicate the position of the movement mechanism where the at least one guide tube and at least one injector tubes are both retracted. The label can indicate the position of the movement mechanism where the at least one guide tube is advanced but the at least one injector tube is retracted. The label can indicate the position of the movement mechanism where the at least one guide tube and at least one injector tube are both advanced. The label can indicate the position of the mechanism to adjust the radial distance to which the at least one guide tube is outwardly expandable. The proximal handle can includes a graphic icon associated with one or more label. The label associated with the position of the mechanism to adjust the radial distance to which the at least one guide tube is outwardly expandable can include a text label, a circular icon, and/or a text label and a circular icon

The mechanism can adjust the pre-set radial distance from the outer surface of the distal portion of the catheter body to which the at least two guide tubes move outward. The mechanism can adjust the pre-set radial distance of three guide tubes. The mechanism can adjust the pre-set radial distance of all of the guide tubes simultaneously.

The method of use can include advancing into the vessel the catheter as described herein. The method can include determining the diameter of the vessel to be treated. The diameter of the vessel can be determined before advancing the catheter or while the catheter is within the patient. The user can use the mechanism to adjust the radial distance to which the at least one guide tube is outwardly expandable with respect to the catheter body to select an appropriate radial distance for the measured vessel diameter. The mechanism can be adjusted before advancing the catheter or while the catheter is within the patient.

The method of use can include operating the longitudinal movement mechanism to advance a pre-set distance at least one guide tube away from the catheter body until the distal end of the at least one guide tube is in proximity to the inside wall of the vessel. The pre-set distance can be determined by the mechanism that adjusts the radial distance. The mechanism to adjust the radial distance to which the at least one guide tube is outwardly expandable with respect to the catheter body has at least two preset selectable settings. The method of use can include operating the longitudinal movement mechanism to extend the at least one injector tube a preset distance beyond the distal end of at least one guide tube, causing the at least one injector tube to penetrate through the inside wall of the target vessel placing the fluid egress of the at least one needle into a volume of tissue outside of the inside wall of the target vessel.

The method of use can include attaching a fluid source to the catheter. The fluid source can be a vial of ablative fluid provided in a kit with the catheter. The kit can include a syringe for injecting the fluid. The syringe can inject fluid into a port and through the check valve. The method of use can include injecting fluid through the catheter injection lumen and out of the needle fluid egress into a volume of tissue outside of the inside wall of the vessel. In some methods, the needles inject fluid. In some methods, the needles are distal electrodes.

The internal mechanism to make the adjustment for vessel size function in the handle 300 requires that the slider 350 control the longitudinal movement of the outer tube 82 with respect to the tapered section 204. When the slider 350 is in the small vessel distal position as shown in FIG. 5A, the outer tube 82 sits in a more distal position as seen with the marker band 325 so the guide tubes 115 of FIG. 4 will be more retracted proximally from the openings 131 in the outer body of the PFIC 150 of FIG. 4. Since the guide tubes 115 start further back proximally, with the same relative advancement of the slider 224 that controls the longitudinal movement of the guide tubes 115 with respect to the opening 131, the guide tubes 115 will not extend as far out of the opening 131. Because only the outer tube 82 is affected, the injector tube 116 with distal needles 119 will still extend the same about beyond the distal end of the guide tubes 115.

It is also envisioned that rather than a slider 350, a lock/unlock button (not shown) could be added to the handle 200 of FIG. 3 that in an unlocked configuration allows the outer tube 82 to be moved longitudinally with respect to the tapered section 204 and when released, will lock the outer tube 82 position with respect to the tapered section 204. The actual total distance that the outer tube 82 would need to move longitudinally to accommodate two ranges can be between 1 and 4 mm with a some embodiments having a distance of 2-3 mm.

FIG. 6A is a cross sectional view of the distal portion of the PTAC 300 guide tube 315 and injector tubes 316 with distal non coring needles 319 in the expanded state. The guide tube 315 with distal end 325 includes an inner layer 323, outer layer 324 and radiopaque marker band 322. The injector tube 316 includes a radiopaque wire 311 and distal fluid egress 317.

FIG. 6B is a cross sectional view of an embodiment of the PFIC 400 injector tube 416 with distal needle 419 designed to function as a single expandable arm 410 without a separate guide tube for deployment of the injection needle 419. The injector tube 416 includes a radiopaque wire 411 and distal fluid egress 417. The injector tube further has a widened section 415, with distal end 425 and radiopaque band 422. One can envision the injector tube 416 with widened section 415 being expanded outward from the body of the PFIC 400 through openings similar to the opening 131 shown in FIG. 4 for the PFIC 150. The tapered shape of the widened section 415 allows the injector tube 416 with widened section 415 to easily slide back into the openings 131 of FIG. 4 when the injector tubes 116 are retracted back into the outer tube extension 104 of the PFIC 400.

The PFIC 400 can have a simpler operation than the PFIC 150 of FIG. 4 as rather than advancing guide tubes 115 then injector tube/needles 116/119 there is only one element to advance outward. In this embodiment of the PFIC 400 it is envisioned that the wall thickness of the injector tube 416 can be uniform as shown in FIG. 6B or could be thicker in the section proximal to the distal end 425 of the thickened section 422 to strengthen it. The material for the injector tube 416 can be a memory metal such as NITINOL. The widened section 415 can be formed from a plastic of relatively low durometer making the distal end 425 soft enough to not cause damage to the inside wall of the target vessel when it engages the wall. The widened section 415 can optionally have a hydrophilic coating to make the widened section 415 slide easily through the openings 131 of FIG. 4.

The radiopaque band 422 and wire 411 can be formed of a radiopaque material such as gold, tantalum or platinum. It is also envisioned that the widened section 422 could be formed from an impregnated plastic such as tungsten filled urethane that can by itself be radiopaque and eliminate the need for the radiopaque band 422.

FIG. 6C is a cross sectional view of an embodiment of the PFIC 500 that integrates together the guide tube and injector tube of the PTAC 300 into a single arm 510 designed to function as a single expandable element without a separate guide tube for deployment of the injection needle 519. The arm 510 has a proximal tubular section 515 with inner layer 523 with lumen 513, outer layer 524 and radiopaque band 533 placed around the outer layer 524. The arm 510 has a distal injector tube section 516 that includes a lumen 521 with radiopaque wire 511 and distal fluid egress 517. The wire 511 is connected to the proximal end of distal injector tube section 516 with a weld 512. Instead of a weld, other means of attachment are envisioned including the use of a solder joint or adhesive.

The injector tube section 516 proximal portion is attached coaxially inside the distal portion of the inner layer 523 of the tube 515. One can envision the arm 510 being expanded outward from the body of the PFIC 500 through openings similar to the opening 131 shown in FIG. 4 for the PFIC 150. The PFIC 500 can have a simpler operation than the PFIC 150 of FIG. 4 as rather than advancing guide tubes 115 then injector tube/needles 116/119 there is only one element, the arm 510 to advance outward. The material for the injector tube 516 can be a memory metal such as NITINOL. The tubular section 515 can be formed either from a memory metal such as NITINOL or from a plastic material. The radiopaque band 522 and wire 511 can be formed of a radiopaque material such as gold, tantalum or platinum. It is also envisioned that either or both inner layer 523 or outer layer 524 of the tubular section 515 could be formed from an impregnated plastic such as tungsten filled urethane that can by itself be radiopaque and eliminate the need for the radiopaque band 522.

The lumen 513 of the inner layer 523 is in fluid communication with a fluid injection lumen similar to the lumen 133 of the PFIC 150 of FIG. 4. The lumen 513 is also in fluid communication with the lumen 521 of the injector tube section 516 which lumen 521 is in fluid communication with the fluid egress 517.

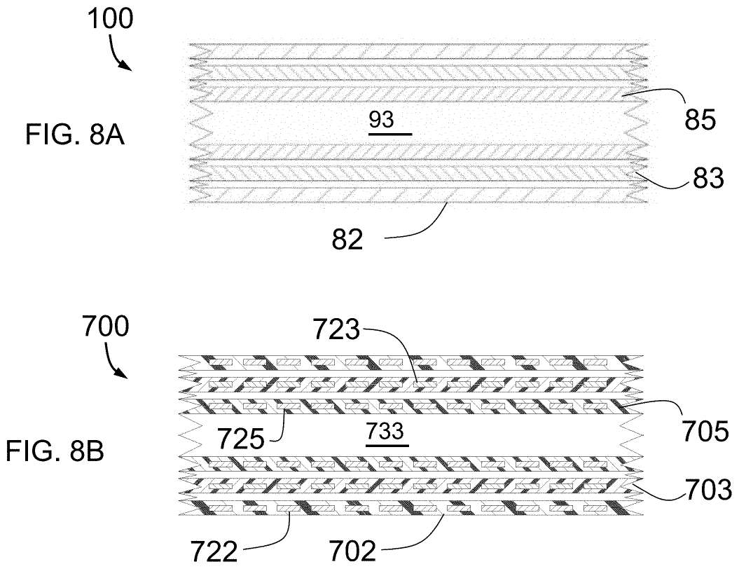

FIG. 7A is a cross sectional view showing the engagement of the distal end 325 of the PTAC 300 guide tube 315 with the inside wall of a target vessel for a vessel smaller than the maximum range of vessels for the PTAC 300. Because of the limitation of proposed radii of curvature for the injector tube 316, the design of the PTAC 300 is constrained if the plane intersecting the distal end 325 of the guide tube 315 is perpendicular to the axis of the guide tube. The angle A shows the angle of the distal end 325 of the guide tube 315 with respect to the inside wall of the target vessel. If the PTAC 300 is designed to have the angle A be zero degrees at the maximum allowable diameter vessel diameter, then for smaller vessels as demonstrated by FIG. 7A, the engagement will produce an angle greater than zero degrees. This can be a particular issue for a PFIC designed to operate in 2 or more vessel ranges.

FIG. 7B is a cross sectional view showing the engagement of the PFIC 600 with guide tube 615 having angulated distal end 625 designed to perform better over a wider range of vessel sizes for use either in a small vessel version of the PFIC 600 that engages with the inside wall of a target vessel. The PFIC 600 is shown with the plane intersecting the distal end 625 of the guide tube 615 having an angle B with respect to the plane perpendicular to the longitudinal axis of the guide tube 615. This angulated distal end 625 may perform better over a wider range of vessel sizes than the non-angulated distal end 325 of the PTAC 300. The angle B can be between 5 degrees and 45 degrees.

It is also envisioned that instead of a dual range version of the PFIC 600, the angulated guide tube 615 could be utilized for a single range small vessel implementation where the only 2 changes required from the PTAC 300 of FIG. 7A using the handle 200 of FIG. 3, are:
1. lengthening of the outer tube 82 of FIG. 3 to position the guide tubes 615 further back in the body of the catheter so they do not extend as far when advanced beyond the openings 131 and
2. using the angulated guide tube 615 so that the angle of engagement with the inside wall of the target vessel B is closer to zero degrees than if one used the guide tube 315 of FIG. 7A designed for a larger range of vessel sizes.

The PFIC embodiments 400, 500 and 600 can be packaged in their deployed configuration so as to help avoid resetting the proper radius of curvature for the plastic components such as the tube 515 of FIG. 6C and the guide tube 615 of FIG. 7B.

FIG. 8A is a longitudinal cross section of a central portion of the PTAC 100 showing the three concentric hypotubes, the inner tube 85 with lumen 93, the middle tube 83 and outer tube 82. While there is an advantage in using metal hypotubes because of their strength and pushability, such metal tubes in the PTAC 100 may require the device be packaged as in a straight configuration making the device package more than a meter in length which means it cannot be easily stored on a shelf. Long package length can be even more of a problem for a radial artery access version that is typically more than 20 cm longer than a femoral access version of the PTAC 100.

While the inner tube 85 must remain as a sealed tube for fluid delivery, it is envisioned that adding holes or slots (e.g. by laser cutting) in the outer and middle tubes 82 and 83 could significantly increase the overall flexibility of the central section of the catheter while maintaining good pushability.

FIG. 8B is a longitudinal cross section of a central portion of an embodiment of PFIC 700 showing the three concentric tubes, an inner tube 705 with fluid injection lumen 733, a middle tube 703 and an outer tube 702. The tubes 702, 703 and 705 are composed a plastic with an integral helically wound flat metal wire. The outer tube 702 has flat wire helix 722, the middle tube 703 has flat wire helix 723 and the inner tube has flat metal wire helix 725. Certain features and uses of helical wound flat wire impregnated tubing is described in Fischell et al. U.S. Pat. No. 8,591,495, issued Aug. 24, 2011; U.S. Pat. No. 8,248,925, issued Jan. 19, 2016; U.S. Pat. No. 5,180,376, issued Jan. 19, 1993; U.S. Pat. No. 5,423,774, issued Jun. 13, 1995, and U.S. Pat. No. 5,484,425, issued Jan. 16, 1996, which are hereby incorporated by reference in their entirety. The use of such tubing here is advantageous as it can allow the PFIC 700 to be packaged in a spiral configuration similar to many existing catheters to fit in a relatively small dimensioned package that could be placed on a shelf.

It is also envisioned that only one or two of the three tubes be formed from a helical wire braided tube and the other could remain as metal hypotubes. It is also envisioned that instead of helical flat wires impregnating the tubes, a wire braid such as that used in guiding catheters could provide for a flexible but pushable tube for use in the PFIC. In any of these configurations, the PFIC 700 can be packaged when wound in a spiral as are many interventional cardiology products such as angioplasty balloons.

FIG. 8C is a longitudinal cross section of a central portion of an embodiment of the PFIC 800 showing metal hypotubes 502P, 802D, 803P, 803D, 805P and 805D flexible connecting tubes 802, 813 and 815 including an inner connecting tube 815, a middle connecting tube 813 and an outer connecting tube 812. The outer connecting tube 812 provides a flexible connection between the proximal outer hypotube 802P and the distal outer hypotube 802D. The middle connecting tube 813 provides a flexible connection between the proximal middle hypotube 803P and the distal middle hypotube 803D. The inner connecting tube 815 provides a flexible connection between the proximal inner hypotube 805P and the distal inner hypotube 805D. It is important that the inner connecting tube 815 provide a fluid seal so that fluid injected into the injection lumen 833 will flow without leaking from the inner proximal hypotube 805P through the inner connecting tube 815 into the distal inner hypotube 805D. A perfect seal is less important for the inner and outer tubes and it is envisioned that instead of connecting the proximal and distal section of middle and outer tube with a connecting tube one or more wires could be welded to provide a flexible but pushable link between the proximal and distal sections of the middle and outer tubes. This also applies to the PFIC 900 of FIG. 8D

In one embodiment the connecting tubes 812, 813, and 815 are formed from a low durometer plastic and several such flexible connectors could be placed along the length of the PFIC 800 to allow it to be placed in a package where it is wound in a spiral as are many interventional cardiology products such as angioplasty balloons. Instead of plastic connecting tubes it is also envisioned that a memory metal such as NITINOL which is non-kinking and extremely flexible could be used for the connecting tubes. As with the PFIC 700 in FIG. 8B, it is envisioned that one or two of the metal hypotubes might remain as metal with only one being split with a flexible connector between proximal and distal sections.

FIG. 8D is a longitudinal cross section of a central portion of an embodiment of the PFIC 900 showing flexible connecting tubes 912, 913 and 915 with integral helical flat wires similar to the tubes 702, 703 and 705 of FIG. 8B. Specifically, these are an inner connecting tube 915 with flat wire helix 925, a middle connecting tube 913 with flat wire helix 923 and an outer connecting tube 912 with flat wire helix 922. The outer connecting tube 912 provides a flexible connection between the proximal outer hypotube 902P and the distal outer hypotube 902D. The middle connecting tube 913 provides a flexible connection between the proximal middle hypotube 903P and the distal middle hypotube 903D. The inner connecting tube 915 provides a flexible connection between the proximal inner hypotube 905P and the distal inner hypotube 905D. It is important that the inner connecting tube 915 provide a fluid seal so that fluid injected into the injection lumen 933 will flow with leaking from the inner proximal hypotube 905P through the inner connecting tube 915 into the distal inner hypotube 905D.

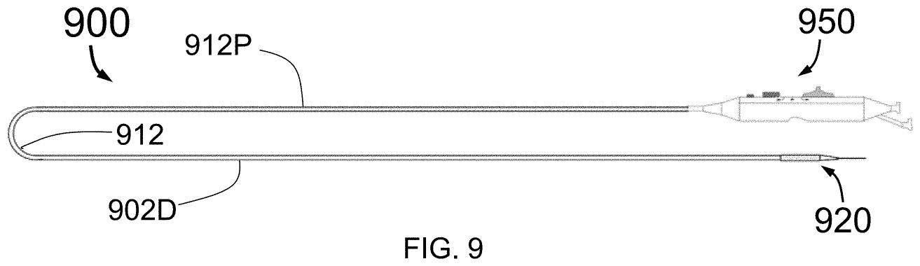

Certain embodiments are taught in the Fischell et al. U.S. Pat. No. 8,591,495, issued Aug. 24, 2011; U.S. Pat. No. 8,248,925, issued Jan. 19, 2016; U.S. Pat. No. 5,180,376, issued Jan. 19, 1993; U.S. Pat. No. 5,423,774, issued Jun. 13, 1995; and U.S. Pat. No. 5,484,425, issued Jan. 16, 1996, incorporated by reference herein. Tubing with a flat wire helix will resist kinking and it is envisioned that having a single set of connecting tubes 912, 913 and 915 of sufficient length could allow the PFIC 900 to be bent in half as seen in FIG. 9 for packaging into a box approximately half the length of certain embodiments of the PTAC 100 which uses three metal hypotubes.

As with the PFIC 700 in FIG. 8B, it is envisioned that one or two of the flexible connecting tubes 912, 912 and 915 might be omitted and the entire length remain as a metal hypotube. It is also envisioned that a wire braid such as is used in guiding catheters could replace the helically wound flat wires 922, 923 and 925 of the tubes 912, 913 and 915 to provide flexibility.

FIG. 9 is a schematic view showing the folded PFIC 900 as it might be positioned for packaging. The PFIC 900 has a handle 950 that can be based on either the handle 200 of FIG. 3 or the PFIC 300 dual range handle 210 of FIGS. 5A and 5B. The distal portion 920 of the PFIC 900 may be similar to the distal portion of the PTAC 100 of FIGS. 1 and 2 or it may include any of the features of the PTAC 300 of FIG. 6A or the PFIC 150, 400, 500, or 600 of FIGS. 4, 6B, 6C and 7B.

The majority of the length of the PFIC 900 comprises the proximal outer hypotube 902P and the distal outer hypotube 902D. These are connected to each other as shown in FIG. 8D with the outer flexible connecting tube 912. The middle connecting tube 913 and inner connecting tube 915 are internal to the catheter and not visible in this view.

Various other modifications, adaptations, and alternative designs are, of course, possible in light of the above teachings. Therefore, it should be understood at this time that within the scope of the appended claims may be practiced otherwise than as specifically described herein.

While this specification has focused on use of the PFIC for use in ablation of tissue, it is also clearly envisioned that the apparatus and methods of FIGS. 1-9 inclusive can be applied to inject any fluid for any purpose including that of local drug delivery into a specified portion of a blood vessel or the volume of tissue just outside of a blood vessel, or into prostatic tissue via the prostatic urethra.

While the embodiments shown in FIGS. 1-9 show three injection needles, the presently disclosed structure which includes radial and/or lateral support mechanisms for needle guiding elements that guide injection needles as they penetrate the interior wall of a target vessel can be applied to designs with one needle, two needles or 5 or more needles.

It is contemplated that various combinations or subcombinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present embodiments herein disclosed should not be limited by the particular disclosed embodiments described above. Moreover, while the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “expanding a balloon” include “instructing the expanding of a balloon.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. A catheter for fluid delivery to a volume of tissue in outside of the inside wall of a target vessel in a human body comprising:
a catheter body comprising a catheter fluid injection lumen;
a central axis extending in a longitudinal direction;
a distal portion comprising at least one guide tube comprising a distal end, the at least one guide tube configured to be outwardly expandable in the radial direction beyond the outer surface of the catheter body with the distal end in proximity to the inside wall of the target vessel;
at least one sharpened needle comprising a needle fluid injection lumen in fluid communication with the catheter fluid injection lumen, a portion of the at least one sharpened needle located coaxially inside of the at least one guide tube, and
a proximal handle comprising an injection port in fluid communication with the catheter fluid injection lumen, the injection port comprising a check valve, the proximal handle configured to advance and retract the at least one guide tube and the at least one sharpened needle.

2. The catheter of claim 1 comprising three guide tubes and three sharpened needles.

3. The catheter of claim 1 wherein the at least one sharpened needle is hollow and comprises a fluid egress near the distal end of the at least one sharpened needle and the catheter fluid injection lumen in fluid communication with the fluid egress of the at least one sharpened needle.

4. The catheter of claim 1 wherein the proximal handle includes at least one indicia associated with the state of the catheter selected from the group consisting of:
a. the position of the movement mechanism wherein the at least one guide tube and at least one injector tubes are both retracted,
b. the position of the movement mechanism wherein the at least one guide tube is advanced but the at least one injector tube is retracted, and
c. the position of the movement mechanism wherein the at least one guide tube and at least one injector tube are both advanced.

5. The catheter of claim 1 wherein the fluid is ablative fluid and wherein the check valve is integrated with the proximal handle and configured to prevent the ablative fluid from flowing back out of the injection port.

6. The catheter of claim 1 wherein the check valve is integrated with the proximal handle and configured to prevent air from entering the catheter fluid injection lumen.

7. The catheter of claim 1 wherein the check valve is integrated with the proximal handle and configured to prevent blood from flowing back through the catheter.

8. A catheter for fluid delivery through at least two injection needles into tissue outside of the interior wall of a target vessel of a human body, the catheter comprising:
a catheter body comprising an outer surface, a central axis extending in a longitudinal direction, and a fluid injection lumen;
at least two guide tubes configured to advance distally and expand outwardly toward the interior wall of the target vessel;
a support structure;
at least two injector tubes with distal injection needles, each of the distal injection needles comprising an injection lumen in fluid communication with the fluid injection lumen of the catheter body, the at least two injector tubes with distal injection needles configured to be advanced outwardly, guided by the at least two guide tubes to penetrate the interior wall of the target vessel, the injection needles comprising a distal opening for fluid delivery into the tissue outside of the interior wall of the target vessel; and
a check valve positioned near the proximal end of the fluid injection lumen.

9. The catheter of claim 8 wherein the support structure comprises a deflection surface, the deflection surface configured to deflect the distally moving guide tubes outward to a pre-set radial distance from the outer surface of the distal portion of the catheter body.

10. The catheter of claim 8 comprising three guide tubes.

11. The catheter of claim 8 wherein the at least one distal injection needle is hollow and includes fluid egress near the distal end of the injection needle and the fluid injection lumen of the catheter body is in fluid communication with the fluid egress of the at least one injection needle.

12. The catheter of claim 8 wherein the check valve is located at the proximal end of the catheter body.

13. The catheter of claim 8 wherein the catheter body comprises an injection port at the proximal end of the fluid injection lumen and the check valve is attached to a proximal end of the injection port.

14. The catheter of claim 8 wherein the check valve is integral to the proximal portion of the fluid injection lumen.

15. A catheter comprising:
a catheter body comprising a fluid injection lumen;
at least one guide tube comprising a distal end, the at least one guide tube moveable between a first position within the catheter body and a second position inclined away from the catheter body, wherein the at least one guide tube is configured to be positioned with the distal end in proximity to an inside wall of a target vessel;
at least one penetrator comprising an injection lumen in fluid communication with the fluid injection lumen of the catheter body, the at least one penetrator configured to penetrate the inside wall of the target vessel, a portion of the at least one penetrator located coaxially inside of the at least one guide tube;
a proximal handle configured to advance and retract the at least one guide tube and the at least one penetrator, and
a check valve in fluid communication with the fluid injection lumen of the catheter body.

16. The catheter of claim 15 wherein the check valve is integrated into the proximal handle.

17. The catheter of claim 15 wherein the check valve is integrated into a fluid injection port in a proximal portion of the catheter.

18. The catheter of claim 17 wherein the fluid injection port includes a non-Luer connector.

19. The catheter of claim 15 further includes a vial of ablative fluid and at least one syringe.

20. The catheter of claim 15 wherein the check valve is configured to allow ablative fluid to flow in one direction from a proximal injection port to an egress of the at least one penetrator and prevent ablative fluid from flowing in the opposite direction out of the proximal injection port.

21-60. (canceled)
